SEQUENCE LISTING (1 sequence)

SEQ ID NO: 1 - DNA, 603 bp, Aequorea victoria
atgaagctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

(19) United States
(12) Patent Application Publication
(10) Pub. No.: US 2008/0003569 A1
(43) Pub. Date: Jan. 3, 2008

(54) Method for the Detection of Intracellular Parameters With Luminescent Protein Probes for the Screening of Molecules Capable of Altering Said Parameters

(76) Inventors: Rosario Rizzuto, Padova (IT); Tullio Pozzan, Venezia (IT); Paolo Pinton, Padova (IT); Carlotta Giorgi, Mantova (IT)

Correspondence Address: ARENT FOX LLP, 1050 CONNECTICUT AVENUE, N.W., SUITE 400, WASHINGTON, DC, 20036, US

(21) Appl. No.: 10/594,298

(22) PCT Filed: Mar. 23, 2005
(86) PCT No.: PCT/IB05/00795

§ 371 Date: Jul. 16, 2007

(30) Foreign Application Priority Data
Mar. 26, 2004 (IT) MI2004A000598

(51) Int. Cl. C12Q 1/68; G01N 33/53

(52) U.S. Cl. 435/6; 435/7.1

(57) ABSTRACT

The invention relates to a method for the detection of intracellular parameters by means of luminescent recombinant protein probes for the screening of molecules capable of altering said target intracellular parameters.

The present invention relates to a method for the detection of intracellular parameters by means of luminescent recombinant protein probes, for the screening of molecules capable of altering said parameters.

In particular, the present invention relates to a method for detecting intracellular parameters and/or activities by means of luminescent protein probes for the screening of molecules of pharmacological and/or cosmetic and/or environmental interest capable of altering said parameters and/or activities.

It is known that cells forming the organism have a continuous exchange of information, both with other cells and with the extracellular environment.

Communication between the cells is guaranteed by their capacity to receive and send signals of a chemical nature interacting with specific receptors present on their plasmatic membrane. In most cases, the activation of these receptors causes the generation of intracellular messengers, called second messengers, which convey the information brought by the extra-cellular messenger (or first messenger) into the cell. With respect to the extreme variety of extra-cellular mediators, the number of known second messengers is surprisingly limited: the calcium ion (Ca²⁺), inositol 1,4,5 triphosphate (IP₃), diacylglycerol (DAG), nucleotides of adenosine and cyclic nucleotides (cAMP and cGMP).

These second messengers in turn activate a series of intracellular performers, called cellular response effectors, which, through a variation in the intracellular parameters, are capable of triggering a series of events until a definitive cellular response is obtained.

Cellular parameters are those values, such as concentration, activation state, cellular localization, which enable us to understand and consequently describe the activity exerted by each single element present inside the cell (ions, proteins, nucleotides, signal molecules).

Variation of said cellular parameters refers to a modification in the normal basic indexes (variation in concentration, translocation to the plasmatic membrane, activation or inactivation), in response to certain stimuli (physiological or pharmacological) deriving from the extra-cellular environment.

Environmental, chemical, physical or genetic factors can create pathological conditions which cause alterations of the transduction pathways of the cellular signal.

In order to understand the functioning of a drug, it is necessary to avail of methods which allow the accurate monitoring of the various intracellular parameters on which the drugs and their variations must act.

At present, there are techniques for detecting only some of the cellular mediators involved in the transduction pathways of the signal and in most cases, these techniques are not efficient and/or easily applicable to massive screening methods of thousands of molecules, a strongly felt requirement, on the other hand, on the part of pharmaceutical, chemical or cosmetic industries.

The most widely-studied cellular mediator was and still is the calcium ion (Ca²⁺). It has been seen that a wide variety of stimuli, ranging from growth factors to neuro-transmitters, are transmitted inside the cell by means of variations in the cytosol concentration of Ca²⁺ ([Ca²⁺]_{c}).

Ca²⁺ is an ubiquitous ion normally present in all cellular compartments, which, through variations in its concentration, is capable of regulating numerous cellular functions.

The development of the first techniques for measuring the concentration of Ca²⁺ goes back to the sixties' and represented the start of a detailed understanding of the mechanisms that regulate [Ca²⁺]_{c} and its role in controlling the numerous biological functions.

The first [Ca²⁺] measurements were effected by micro-injecting into huge cells of Balanus, a Ca²⁺-sensitive photo-protein, aequorin (Ridgway and Ashley, 1967); in subsequent years, metallochromic indicators and specific micro-electrodes were often used. In both cases, the techniques were limited by the necessity of micro-injecting the indicator or inserting an electrode into the cell and consequently by the possibility of using few large dimensional cellular types.

In the nineties', the use of fluorescent Ca²⁺ indicators such as, for example, quin2 and fura-2, capable of passing through the plasmatic membrane and remaining entrapped at a cytoplasmatic level (Tsien et al., 1982, Grynkiewicz et al., 1985), allowed the cellular homeostasis of Ca²⁺ to be studied in many cellular types and to demonstrate the ubiquitous role of this ion. The facility of use of fluorescent indicators, however, is opposed by the incapacity of being selectively accumulated in the various cellular organelles.

The above techniques, however, cannot be easily applied to pharmacological screening studies and are in any case limited to the measuring of Ca²⁺ alone.

The arrival of molecular biological techniques contributed, in the nineties', to the development of new methods for monitoring the Ca²⁺ ion based on the construction of protein probes which are introduced inside the cells by means of transfection techniques. There are currently two categories of recombinant probes for Ca²⁺.

The first group exploits the fluorescent characteristics of molecules deriving from the fluorescent protein GFP (Green Fluorescent Protein, Zhang et al., 2002). These probes are difficult to use for pharmacological screening as they require extremely costly procedures in terms of time and the ratio between the fluorescent signal obtained under standby and activation conditions is not sufficiently wide to allow use for automated methods.

The second category of probes, is based on the use of bioluminescent proteins capable of binding the Ca²⁺ and consequently emitting a luminous radiation, which can be measured and correlated to the concentration of the Ca²⁺ itself, among which aequorin.

Aequorin, extracted and purified in 1962 from a species of luminescent medusa, Aequorea Victoria (Shimomura et al., 1962), is a protein of about 22 kDa consisting of an apoprotein and a hydrophobic prosthetic group, celentherazine, bound to the apoprotein by means of a covalent link of the peroxide type.

The Ca²⁺ link with aequorin at a level of 3 specific high affinity sites of the “EF-hand” type, induces a conformational modification of the protein itself and consequent breakage of the covalent link and emission of photons and release of the oxidized coenzyme.

It is possible to measure the variation in the [Ca²⁺] thanks to the existence of a relation between the logarithm of the emission rate of photons (L), expressed as a fraction with respect to the maximum luminescence rate, i.e. under saturation conditions (L/Lₘₐₓ) and the logarithm of [Ca²⁺]. These values, obtained through in vitro measurements, under known conditions of pH, ion strength [Mg²⁺] and temperature, allow a sigmoid-shaped calibration curve to be constructed, on the basis of which, within the range of Ca²⁺ concentrations from 0.1-μM (and over 100 μM using mutated aequorin (Montero et al., 1995), it is possible to correlate, at each moment, the fraction of aequorin consumed with the [Ca²⁺] value at which the photo-protein is exposed. The conversion of the luminous signal obtained from the cells, is based on this relation, in values of [Ca²⁺].

Aequorin, as bioluminescent marker, has advantages with respect to fluorescent indicators as it can be easily directed into the various cellular compartments by the use of appropriate regulating elements or signal peptides, representing a reliable instrument for measuring the variations in the concentration of Ca²⁺ (Rizzuto et al., 1992). In particular, this publication describes the technique for directing and measuring the Ca²⁺ concentrations at a mitochondria level.

Various recombinant probes of aequorin were subsequently developed, directed towards other cellular compartments such as, for example, nucleus (Brini et al., 1993; Brini et al., 1994), endoplasmatic reticulum (Montero et al., 1995), Golgi apparatus (Pinton et al., 1998), mitochondria inter-membrane space (Rizzuto et al., 1998), sarcoplasmatic reticulum (Brini et al., 1997), sub-membrane region (Marsault et al., 1997) which made it possible to determine the variations in the concentration of Ca²⁺ in the various cellular districts following various kinds of stimulation.

Another advantage of luminescence is that, with respect to fluorescence, it does not require an excitation light thus avoiding self-fluorescence and photobleaching phenomena. Furthermore, aequorin is not toxic, it does not form links with other cations and does not interfere with the intracellular Ca²⁺ concentration.

The authors previously studied the use of a Ca²⁺ sensitive recombinant photo-protein, such as aequorin, expressed in mammal cells as an alternative method for measuring the concentration of the Ca²⁺ ion (Rizzuto et al., 1993; Rizzuto et al., 1994; Brini et al., 1994b; Rizzuto et al., 1995; Brini et al., 1995; De Giorgi et al., 1996; Rutter et al., 1996; Brini et al., 1999; Robert et al., 2000; Porcelli et al., 2001; Chiesa et al., 2001).

The use of aequorin has a series of technique advantages described hereunder:

a) optimum signal/noise relation as mammal cells do not have endogenous luminescent protein;

b) it integrates the data of a transfected population, thus avoiding errors depending on cellular variability;

c) the aequorin probe can be stably expressed alone in a cellular line (thus obtaining a “permanent” and repeatable screening system), or stably co-expressed with specific receptors of interest (thus simplifying the functional analysis of the specific signaling systems);

d) extremely simple detection system, as it is sufficient to collect the whole light emission spectrum emitted, which is suitable for the miniaturization of the sample to be analyzed and automation, as the luminous signal is strong (the light emission increases logarithmically with the variation in the Ca²⁺ concentration).

It is interesting to observe, however, that although variations in the calcium ion can be easily detected, they are only involved in a few of the numerous signal transduction pathways of pharmaceutical interest. As already specified, there are, however, many other transduction pathways which involve other cellular elements (cellular effectors, enzymes, ion channels, second messengers) which are difficult to detect directly, but which are extremely important for identifying molecules of potential pharmacological or toxicological interest.

In view of what is indicated above, there is evidently the necessity for availing of effective, sensitive and rapid methods for the detection of intracellular parameters and/or activities, for the screening of molecules suitable for generating specific alterations in said parameters and/or activities.

The authors of the present invention have now developed new methods capable of indirectly detecting variations in the intracellular parameters, retracing said variations to corresponding variation of the concentration of the Ca²⁺ ion which can be easily detected. In particular, bioluminescent detections systems have been set up, based on the use of the aequorin photo-protein, capable of emitting luminous signals in response to variations in the concentration of Ca²⁺, which reflect the variation in other intracellular parameters or enzymatic activities in response to certain physiological and/or pharmacological stimulations.

The conversion of intracellular parameters and activities into a variation of the calcium concentration enables both the second messenger generation, such as cAMP, for example, as well as the activation and/or translocation of performer proteins, such as, for example, the kinase proteins (regulating proteins such as PKC) or the proteins of the shc family (protein adaptors such as p46shc, p52shc or p66shc) to be followed.

Among performer proteins of interest, the authors of the present invention examined, for illustrative purposes, the protein kinase C (PKC) and the protein p66shc. As PKC and p66shc, following activation, translocate from the cytoplasm to the plasmatic membrane where the concentration of the calcium ion is greater with respect to the concentration in the cytoplasm, the authors created a PKC/aequorin and p66shc/aequorin chimerical proteins particularly sensitive to the different Ca²⁺ concentration. In this way, the translocation to the membrane and consequent activation of the PKC and p66shc were correlated to an indirect parameter such as the different Ca²⁺ concentration in the two cellular areas (cytoplasm and sub-membrane area).

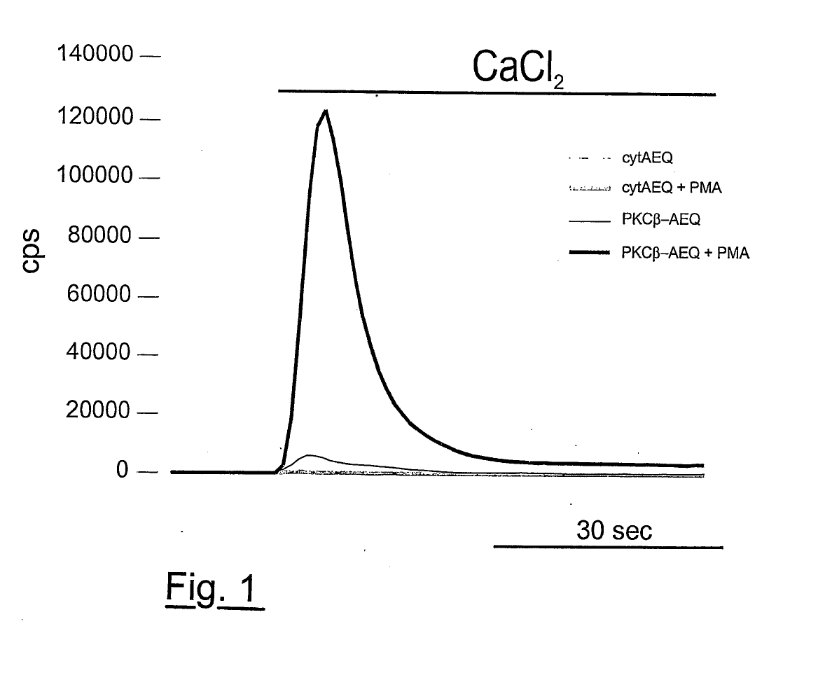

The authors show that the luminous signal of PKC-aequorin and p66shc-aequorin at the level of the membrane is (due to the non-linearity of the luminescence function with respect to the [Ca²⁺]) at least 50-100 times more intense with respect to that of a PKC-aequorin and p66shc-aequorin probe at a non-activated cytoplasmatic level. The luminescent signal of chimerical proteins considered is much more intense after induction of translocation with a known activator, as is shown in FIGS. 1-6 relating to the experiments effected with a prototype of these chimerical proteins.

The method identified by the authors allows the translocation/activation of the proteins to be simply, economically and efficiently quantified, as the light emission increases proportionally with the translocation degree, and a screening of compounds, active on said proteins, to be effected.

In parallel, the authors of the present invention applied the same innovative system for converting the concentration values of second messengers into Ca²⁺ concentration values, i.e. transforming a signal which is difficult to quantify into a signal that can be easily detected, again using aequorin as detection system.

Among second messengers, the authors of the present invention examined cAMP. In this case, chimerical receptors were developed, in which the intracellular portion of the original receptor was substituted, i.e. the portion capable of activating the cellular response through the production of cAMP, with the intracellular portion of a receptor (coupled with Gq proteins) capable of inducing a variation in the concentration of Ca inside the cell. In this way, once the chimerical receptor has been stimulated, it will induce increases in the concentration of calcium rather than of cAMP.

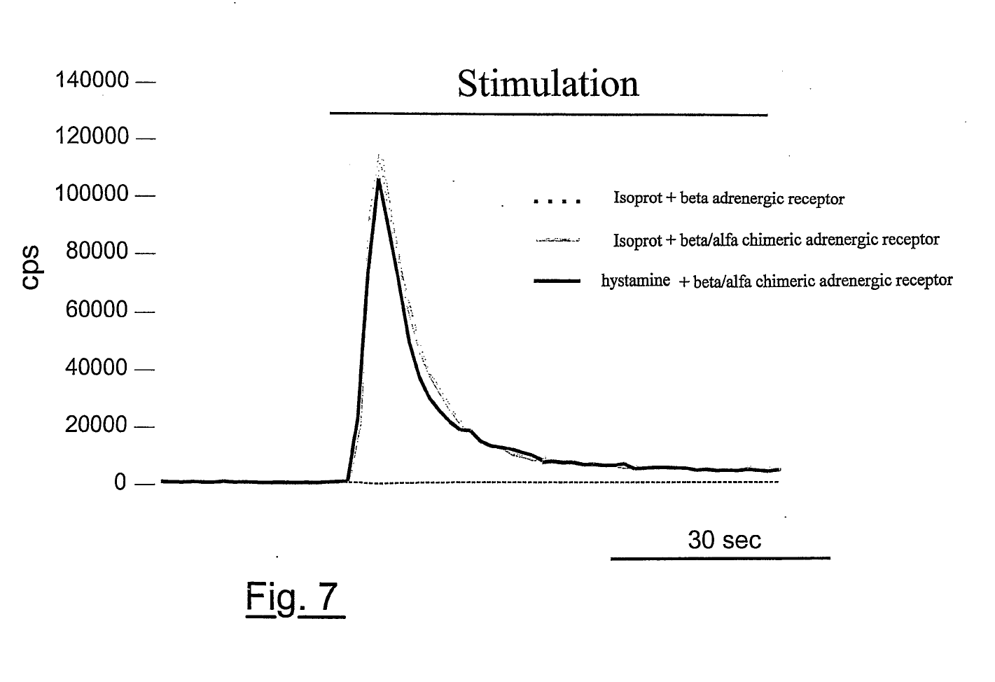

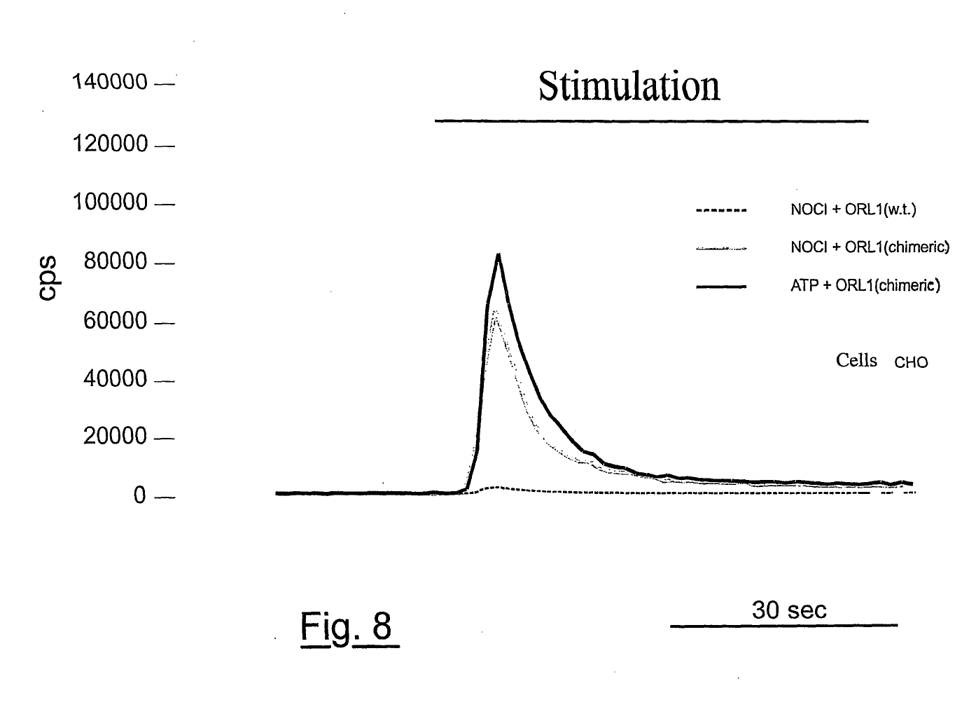

A condensed aequorin was used as detection probe, at a signal frequency that directs it towards mitochondria (mt-AEQ) (Rizzuto et al., 1992), as a result of its greater sensitivity due to the fact that the mitochondria “amplify” the increases in concentration of cytosol calcium. As it can be inferred from FIGS. 7 and 8, the luminescent signal of the mt-AEQ probe is comparable in cells treated with a stimulation normally associated with increases in the concentration of calcium (histamine and ATP) or in cells treated with a stimulation associated with increases in the concentration of cAMP (isoproterenol or nociceptine).

Another application of this type of luminescent probes based on aequorin found by the authors of the present invention is represented by the possibility of analyzing the catalytic activity of cellular effectors (such as PKC) which act on the ionic channels of calcium and identifying drugs which interfere with said catalytic activity. The calcium channels are localized on the membranes of important cellular compartments, such as, for example, the plasmatic membrane, the membrane of the endoplasmatic reticulum or the internal mitochondrial membrane. These are involved in the fine regulation of the intracellular homeostasis of calcium and alterations in their function are associated with pathological conditions.

For this purpose, the authors used an appropriate aequorin probe (variable in relation to the cellular compartment on which the channel of interest is localized) and a cellular line expressing (endogenously or following engineering) the calcium channel positively or negatively regulated by the cellular effector, in the specific case by PKC negatively.

In the system set up by the authors, it is possible to identify the substances which act by inhibiting the catalytic activity of PKC through the detection of an increase in the luminescent signal of aequorin indicating the flow of calcium through the channel which cannot be closed by the kinase enzymatic action.

The various applications of the luminescent probes produced by the authors of the present invention, allow the differentiated screenings to be effected, of the molecules to be tested and as they can be easily automated, they can be used both for HTS (high throughput screening) analyses and for MTS (medium throughput screening) or LTS (low throughput screening) measurements.

The object of the present invention therefore relates to a screening method according to the enclosed claims.

In a preferred embodiment of the present invention, a screening method is provided, for molecules capable of generating the alteration of a target intracellular parameter, said alteration being converted into a proportional variation of the intracellular concentration of the Ca²⁺ ion detected by means of a Ca²⁺-sensitive recombinant protein probe, comprising the following phases:

a) construction of an expression vector containing the sequence encoding said probe, said sequence being characterized in that it comprises sequences encoding a Ca²⁺-sensitive photo-protein, preferably aequorin, and at least one cellular effector or a signal sequence, condensed together;

b) transfection of at least one cellular line of a mammal mal with said vector containing the Ca²⁺-sensitive recombinant protein probe;

c) activation of aequorin by the addition of a prosthetic group, preferably celentherazine, to the cellular line expressing said recombinant protein probe;

d) administration of the molecule to be tested to the cellular line expressing said recombinant protein probe;

e) detection of the emission of photons on the part of the Ca²⁺-sensitive photo-protein, preferably aequorin, expressed in the cellular line.

For greater clarity, the term “Ca²⁺-sensitive photo-protein” refers to any amino acidic sequence capable of emitting photons, following the bond with calcium ions; aequorin and obelin proteins are particularly important from an applicative point of view.

“Performer or effector proteins refers to regulating proteins, proteins which link membrane receptors, proteins which link membrane channels, proteins which link membrane lipids.

“Regulating protein” refers to any amino acidic sequence capable of modifying the activity and/or structure of other cellular components, following its interaction with other signal molecules.

“Proteins that link membrane receptors” refer to those amino acidic sequences capable of interacting with receptors situated at the level of cellular membranes. Among these, it is possible to distinguish adaptors which allow the interaction between the activated receptor and a third protein, and modulators which directly interfere with the activity of the receptor.

The term “proteins that link membrane channels” indicate those amino acidic sequences capable of interacting with channels situated at the level of cellular membranes.

Furthermore, the term “proteins that link membrane lipids” are identified as those amino acidic sequences capable of interacting with lipids situated at the level of cellular membranes.

Finally, the term cellular compartment refers to any region inside the cell preferably delimited by cellular membranes such as: cytoplasm, area below the plasmatic membrane, nucleus, mitochondria, endo-sarcoplasmatic reticulum, Golgi apparatus, vesicles, lyso-endosomes.

The present invention is described hereunder for illustrative but non-limiting purposes, according to its preferred embodiments, with particular reference to the figures of the enclosed drawings, in which:

FIG. 1 shows a graph which illustrates the measuring of the translocation entity (cps) of the probe with the isoform PKCβ-aequorin in the presence or in the absence of PMA stimulation and the difference with respect to the aequorin cytosolic cyt-AEQ probe;

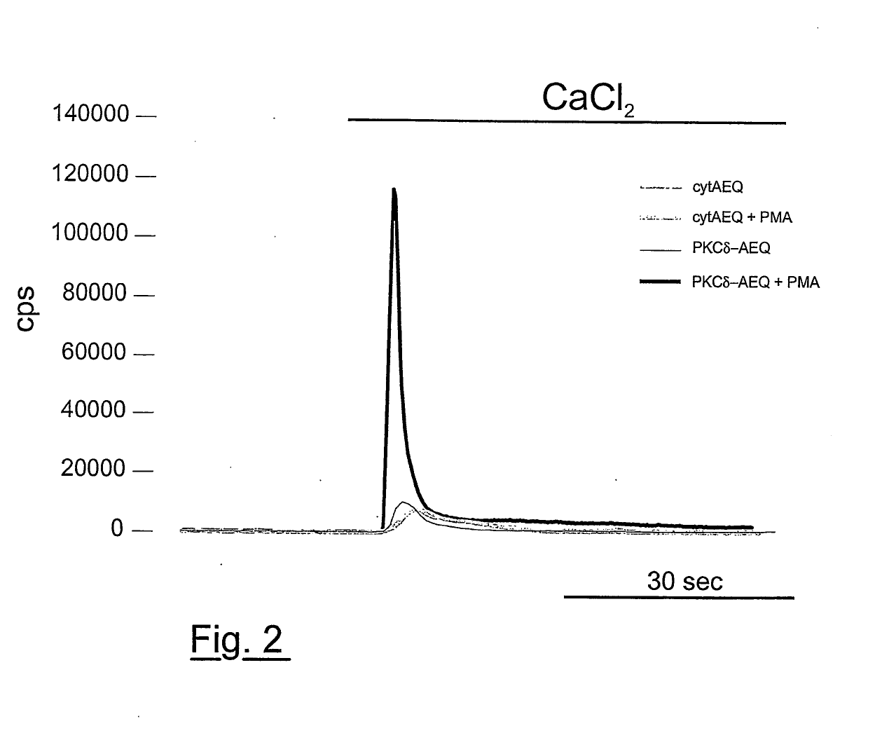

FIG. 2 shows the graph which illustrates the measuring of the translocation entity (cps) of the probe with the isoform PKCδ-aequorin in the presence or in the absence of PMA stimulation and the difference with respect to the aequorin cytosolic cyt-AEQ probe;

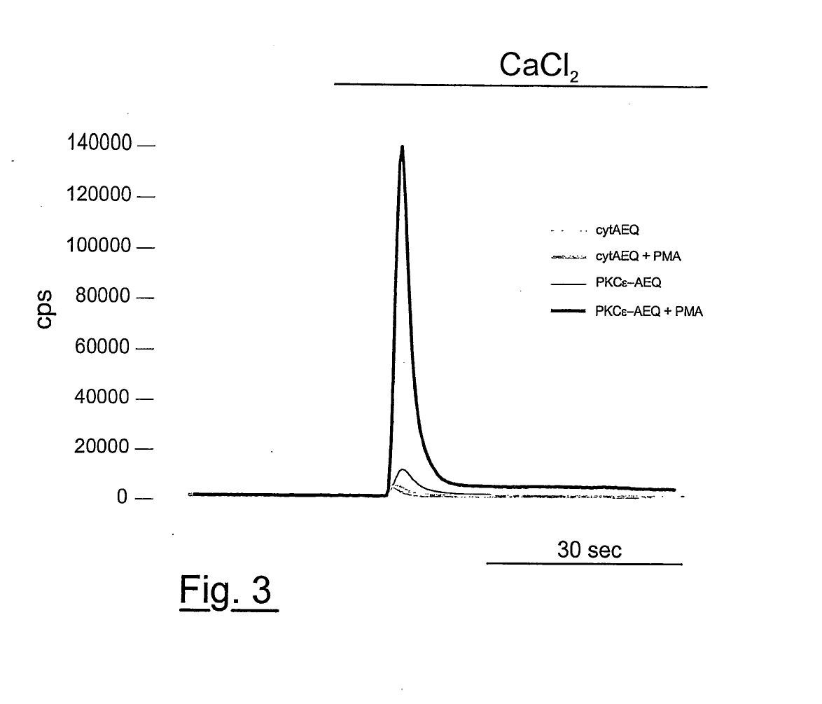

FIG. 3 shows the graph which illustrates the measuring of the translocation entity (cps) of the probe with the PKCε-aequorin isoform in the presence or in the absence of PMA stimulation and the difference with respect to the aequorin cytosolic cyt-AEQ probe;

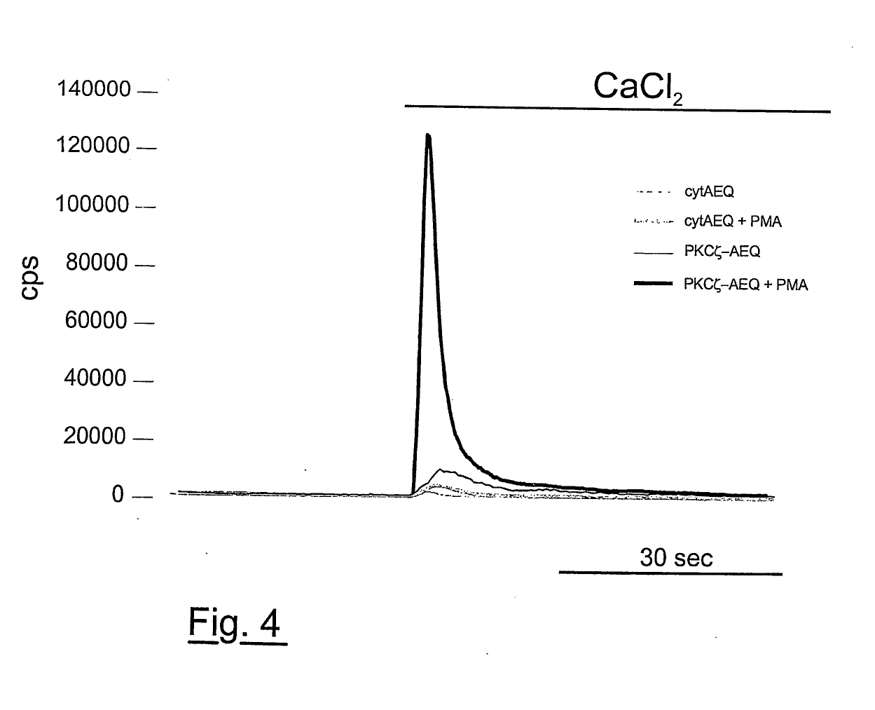

FIG. 4 shows the graph which illustrates the measuring of the translocation entity (cps) of the probe with the PKCζ-aequorin isoform in the presence or in the absence of PMA stimulation and the difference with respect to the aequorin cytosolic cyt-AEQ probe;

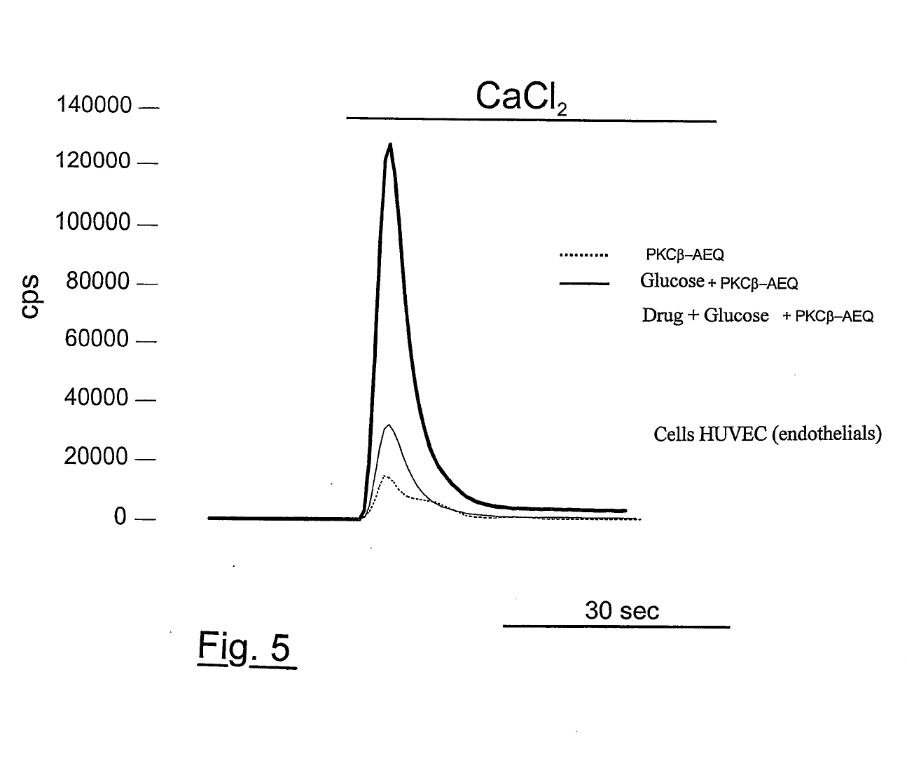

FIG. 5 shows a graph which illustrates the measuring of the translocation entity (cps) of the PKCβ-AEQ probe under hyperglycaemia conditions in the presence or absence of a drug;

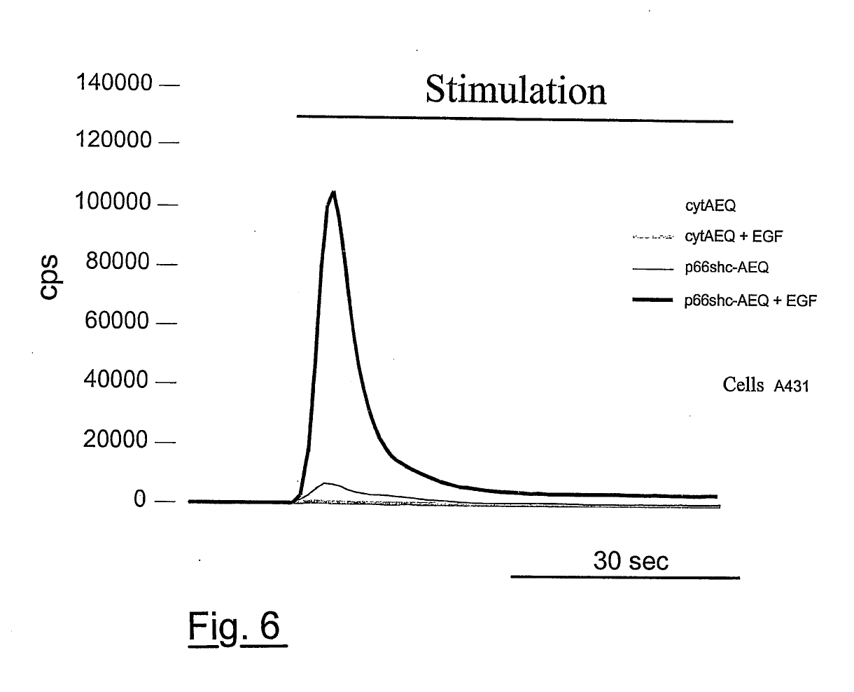

FIG. 6 shows the graph which illustrates the measuring of the translocation entity (cps) of the p66shc-AEQ probe in the presence or in the absence of EGF stimulation and the difference with respect to the aequorin cytosolic cyt-AEQ probe;

FIG. 7 shows the graph which illustrates the measuring of the entity (cps) of the cAMP concentration increase in the presence of the beta adrenergetic receptor and isoprotenol or of the beta/alpha adrenergetic chimerical receptor and isoprotenol or histamine;

FIG. 8 shows the graph which illustrates the measuring of the entity (cps) of the cAMP concentration increase in the presence of the wild-type ORL1 receptor for nociceptine and of nociceptine or of the adrenergetic ORL1/alpha chimeric receptor and of ATP or nociceptine.

### EXAMPLE 1

### Analysis of the Translocation to the Plasmatic Membrane of a Cellular Effector of Interest, the Kinase Protein C (PKC)

Materials and Methods

Construction of a PKC-Aequorin Chimeric Probe

In order to validate our procedure, various types of a series of PKC-aequorin chimeric probes were designed, each containing a different PKC isoform:

1) PKC beta-aequorin (beta PKC: ref. M13975)

2) PKC delta-aequorin (delta PKC: ref. M18330);

3) PKC epsilon-aequorin (epsilon PKC: ref. AF028009);

4) PKC zeta-aequorin (zeta PKC: ref. M18332);

5) PKC gamma-aequorin;

6) PKC alpha-aequorin (alpha PKC: ref. M13973);

7) PKC lambda-aequorin;

8) PKC theta-aequorin (theta PKC: ref. L07032);

9) PKC eta-aequorin.

In particular, the nucleotide sequence of aequorin used for the construction of PKC-aequorin probes is the following:

ATG AAG CTT TAT GAT GTT CCT GAT TAT GCT AGC CTC
AAA CTT ACA TCA GAC TTC GAC AAC CCA AGA TGG ATT
GGA CGA CAC AAG CAT ATG TTC AAT TTC CTT GAT GTC
AAC CAC AAT GGA AAA ATC TCT CTT GAC GAG ATG GTC
TAC AAG GCA TCT GAT ATT GTC ATC AAT AAC CTT GGA
GCA ACA CCT GAG CAA GCC AAA CGA CAC AAA GAT GCT
GTA GAA GCC TTC TTC GGA GGA GCT GGA ATG AAA TAT
GGT GTG GAA ACT GAT TGG CCT GCA TAT ATT GAA GGA
TGG AAA AAA TTG GCT ACT GAT GAA TTG GAG AAA TAC
GCC AAA AAC GAA CCA ACG CTC ATC CGT ATA TGG GGT
GAT GCT TTG TTT GAT ATC GTT GAC AAA GAT CAA AAT
GGA GCC ATT ACA CTG GAT GAA TGG AAA GCA TAC ACC
AAA GCT GCT GGT ATC ATC CAA TCA TCA GAA GAT TGC
GAG GAA ACA TTC AGA GTG TGC GAT ATT GAT GAA AGT
GGA CAA CTC GAT GTT GAT GAG ATG ACA AGA CAA CAT
TTA GGA TTT TGG TAC ACC ATG GAT CCT GCT TGC GAA
AAG CTC TAC GGT GGA GCT GTC CCC TAA.

In the specific example, the results presented relate to the use of the PKC beta-aequorin (PCKβ-AEQ), PKC delta-aequorin (PCKδ-AEQ), PKC epsilon-aequorin (PCKε-AEQ), PKC zeta-aequorin (PCKζ-AEQ) probes.

These probes were obtained by inserting, in a eukaryotic expression vector (pcDNA3; 5.4 kb), the sequence encoding the various PKC isoforms condensed in frame with the sequence encoding aequorin described above.

The localization of the chimeric probe was determined by signal peptides contained inside the native sequence of the protein whose intracellular path is to be followed, in this specific case PKC. The addition of the cDNA of aequorin to the C-terminal of the sequence encoding PKC does not alter either the orientation or the physiological behaviour of the protein being examined.

At this point, transfection is effected in different cellular lines of the chimeric probe obtained, such as HeLa, Cos 7 or Hek 293 cells.

Engineering of the Cellular Line

In the example illustrated, it was not necessary for the cellular line used to express any other element to activate the cellular response. It is possible, however, depending on study requirements, to induce the cells to express a specific receptor for the molecule/drug to be tested in order to ensure an effective linkage degree, thus simplifying the functional analysis of the transduction systems.

Cellular Transfection

The cells cultivated on cellular culture flasks (75 cm²) were transfected with a vector containing the chimeric probe produced, using the most suitable transfection techniques for the cellular line in question. As the HeLa stabilized cellular line was used as experimental model, the calcium phosphate technique was adopted as transfection method which guarantees a high percentage of positive cells for this cellular line.

Collection of Cells Expressing the Chimeric Probe

36 hours after transfection, the cells contained in the flask were detached from the bottom by trypsinization. The cellular suspension was then transferred to a Falcon tube (15 to 50 ml) and subjected to centrifugation at 1200 rpm at 20° C. and finally the cellular precipitate was re-suspended in KRB (Krebs-Ringer modified buffer: 125 mM NaCl, 5 mM KCl, 1 mM Na₃PO₄, 1 mM MgSO₄, 5.5 mM glucose, 20 mM HEPES, pH 7.4, 37° C.).

Reconstitution of the Aequorin Photo-Protein to Active Form

The prosthetic group celentherazine was added to the suspension of cells expressing the PKC-aequorin probe at a final concentration of 5 μM.

Planting on Multi-Well Plates

50 μl of suspension containing transfected cells (corresponding to about 100,000 cells) were planted in each well of the plate. The cells were left to adhere for a time varying from 1 to 2 hours keeping them in the dark, due to the photo-instability of celentherazine.

Detection of the Response

At the end of the incubation time, the various molecules to be tested were added to each well and the plate with the treated cells was then put in direct contact with a photo-multiplier which measures the emission of photons on the part of aequorin.

Results

This simple test can be used for testing compounds capable of modulating the translocation of PKC in live cells.

FIGS. 1-4 show the graphs which indicates the measuring of the translocation to cps of PKC beta, delta, epsilon and delta, according to the method described above.

The examples, shown were carried out on two parallel batches of cells:
a) HeLa cells expressing the PKC-aequorin (PKC-AEQ) probe (dark lines).
These cells were in turn subdivided into:
HeLa PKC-aequorin cells treated with PMA (1 μM, SIGMA) to imitate the action of a drug (thick dark line);
control HeLa PKC-aequorin cells non treated with PMA (thin dark line);
b) HeLa cells expressing the cytosol aequorin probe (cyt-AEQ) (not condensed to any protein) (light lines).
These cells were in turn subdivided into:
HeLa cells with cytosol aequorin treated with PMA to imitate the action of a drug (thick light line);
control HeLa cells with cytosol aequorin non treated with PMA (thin light line).

As can be observed from the trend of the graphs shown in FIG. 1-4, in the absence of external calcium, the emission of photons (expressed as cps: counts per second) has a very low intensity (lower than 100 cps), under all the conditions examined. Under such conditions, in fact, the concentration of calcium in the cytoplasm, where the cyt-AEQ probes (both in the absence and in the presence of PMA forbol ester) and PKC-AEQ probes (in the absence of PMA) are located, and below the plasmatic membrane, i.e. where the PKC-AEQ probe translocates following treatment with PMA, is very low (0.1 μM).

The addition of external calcium induces a significant flow of this ion through the channels situated on the plasmatic membrane with a consequent increase in the concentration of calcium in the region below the plasmatic membrane. Under these conditions, an enormous increase in the emission of photons is registered (>100,000 cps) only in the cells expressing the PKC-AEQ probe, treated with PMA, indicating the complete translocation of the kinase.

In all the other groups of cells, there is no significant variation in the emission of photons (lower than 10,000 cps) on the part of aequorin.

From the graph, it is evident that all the cps values higher than those observed in the absence of the maximal activator (PMA) will indicate a complete translocation of the kinase. With this system, it is therefore possible not only to identify the molecules capable of inducing a translocation, but also to evaluate the amount of activation or inhibition exerted by the molecule tested, on the basis of a ratio between the cps value obtained and the maximum cps value registered under conditions of maximum stimulation of the cellular line.

The translocation efficiency can be determined by expressing it on the basis of a range between the cps value obtained under control conditions (0%) (without PMA) and that obtained following maximal translocation (100%) (after treatment with PMA), which can be described as follows:
GOOD activator for values ranging from 70% to 100% with respect to the maximal activator;
MEDIUM activator for values ranging from 40% to 70% with respect to the maximal activator;
POOR activator for values ranging from 10% to 40% with respect to the maximal activator;
NON-activator for values below 10% with respect to the maximal activator.

This result clearly shows the validity of the system proposed for measuring the translocation of proteins from the cytosol compartment to the plasmatic membrane in response to external stimulations.

### EXAMPLE 2

### Analysis of the Translocation to the Plasmatic Membrane of Beta PKC under Hyperglycaemia Conditions

Materials and Methods

Construction of the Chimeric PKC-Aequorin Probe

A beta-aequorin PKC probe, already described in example 1, was used, with which HUVEC endothelial cells were transfected.

Engineering of the Cellular Line

In the example provided, it was not necessary for the cellular line used to previously express any other element to start the cellular response. It is possible, however, depending on the study requirements, to induce the cells to express a specific receptor for the molecule/drug to be tested in order to assure a sufficient linkage degree, thus simplifying the functional analysis of the transduction systems.

Cellular Transfection

The cells cultivated on cellular culture flasks (75 cm²) were transfected with a vector containing the chimeric probe produced, using the most suitable transfection techniques for the cellular line in question. Endothelial cells of stabilized umbilical cord veins, called HUVEC, were used as the experimental model, which were transfected with the PKC beta-aequorin probe, using lipofectamine which guarantees a high percentage of positive cells for this cellular line.

Collection of the Cells Expressing the Chimeric Probe

36 hours after transfection, the cells contained in the flask were detached from the bottom by trypsinization. The cellular suspension was then transferred to a Falcon tube (of 15 or 50 ml) and subjected to centrifugation at 1,200 rpm and at 20° C., and finally the cellular precipitate was re-suspended in KRB (Krebs-Ringer modified buffer: 125 mM NaCl, 5 mM KCl, 1 mM Na₃PO₄, 1 mM MgSO₄, 5.5 mM glucose, 20 mM HEPES, ph 7.4, 37° C.).

Reconstitution of the Aequorin Photo-Protein to Active Form

The prosthetic group celentherazine at a final concentration of 5 μM was added to the cell suspension expressing the PKC beta-aequorin probe.

Planting on Multi-Well Plates

50 μl of suspension containing transfected cells (corresponding to about 100,000 cells) were planted in each well of the plate. The cells were left to adhere for a time varying from 1 to 2 hours, keeping them in the dark, due to the photo-instability of celentherazine.

Detection of the Response

At the end of the incubation time, the various molecules to be tested were added to each well and the plate with the treated cells was then put in direct contact with a photo-multiplier which measures the emission of photons on the part of aequorin.

Results

This simple test can therefore be used for testing compounds capable of modulating the PKC translocation into the live cells.

FIG. 5 shows a graph which indicates the measuring of the PKC beta translocation, according to the method described above.

The example provided was carried out on three parallel batches of cells:

a) HUVEC cells expressing the PKC beta-aequorin (PKCβ-AEQ) probe, maintained under control conditions (absence of glucose) (dotted line).

b) HUVEC cells expressing the PKC beta-aequorin (PKCβ-AEQ) probe, maintained under hyperglycaemia conditions (10 mM of glucose) (dark, thick line).

c) HUVEC cells expressing the PKC beta-aequorin (PKCβ-AEQ) probe, maintained under hyperglycaemia conditions (10 mM of glucose) treated with a well-known anti-diabetic agent, metformin (20 μM) (dark, thin line).

As can be seen from the trend of the graph shown in FIG. 5, the emission of photons (expressed as cps: counts per seconds), in the absence of external calcium, is of a very low intensity (lower than 100 cps) in all conditions examined. In those conditions, in fact, the calcium concentration in the cytoplasm, where the PKCβ-AEQ probe (in the absence of glucose) is localized, and under the plasmatic membrane, i.e. where the PKCβ-AEQ probe translocates following treatment with glucose, is very low (0.1 μM).

The addition of external calcium induces a sustained flow of said ion through the channels situated on the plasmatic membrane with a consequent increase in the calcium concentration in the region under the plasmatic membrane. Under these conditions, an enormous increase in the emission of photons (>100,000 cps) is registered only in the cells expressing the PKCβ-AEQ probe, treated with high glucose, indicating the completed translocation of kinase under hyperglycaemia conditions.

The cells kept under hyperglycaemia conditions, treated with the anti-diabetic drug show a considerable decrease in the response, in terms of photon emissions (lower than 40,000 cps) which means a good inhibition of the PKCβ-AEQ probe translocation.

In the control cells, no significant variation is observed of the emission of photons on the part of aequorin (lower than 10,000 cps).

From the graph, it appears evident that the treatment of cells kept under high glucose conditions by means of the anti-diabetic drug metformin, inhibits the translocation induced by glucose under hyperglycaemia conditions.

With this system, it is not only possible to identify the molecules capable of inhibiting a translocation, but also to evaluate its efficiency, by expressing it on the basis of a range between the cps value obtained under control conditions (0%) (without glucose) and that obtained following maximal translocation (100%) (after treatment with high glucose), which can be described as follows:
GOOD inhibitor for values ranging from 10% to 40% with respect to the maximal activation;
MEDIUM inhibitor for values ranging from 40% to 70% with respect to the maximal activation;
POOR activator for values ranging from 70% to 100% with respect to the maximal activation.

This result clearly shows the validity of the system proposed for measuring the translocation of proteins from the cytosol compartment to the plasmatic membrane in response to external stimulations.

### EXAMPLE 3

### Analysis of the Translocation to the Plasmatic Membrane of the Protein Adaptor p66 Belonging to the Family of shc Proteins

Materials and Methods

Construction of the Chimeric p66shc-Aequorin Probe

A chimeric probe p66shc-aequorin was designed in order to further validate our procedure.

The nucleotide sequence of the protein p66shc used for the construction of the p66shc-aequorin probe has, as a reference: PUBMED 9049300.

The nucleotide sequence of aequorin used for the construction of p66shc-aequorin probes is the following:

ATG AAG CTT TAT GAT GTT CCT GAT TAT GCT AGC CTC
AAA CTT ACA TCA GAC TTC GAC AAC CCA AGA TGG ATT
GGA CGA CAC AAG CAT ATG TTC AAT TTC CTT GAT GTC
AAC CAC AAT GGA AAA ATC TCT CTT GAC GAG ATG GTC
TAC AAG GCA TCT GAT ATT GTC ATC AAT AAC CTT GGA
GCA ACA CCT GAG CAA GCC AAA CGA CAC AAA GAT GCT
GTA GAA GCC TTC TTC GGA GGA GCT GGA ATG AAA TAT
GGT GTG GAA ACT GAT TGG CCT GCA TAT ATT GAA GGA
TGG AAA AAA TTG GCT ACT GAT GAA TTG GAG AAA TAC
GCC AAA AAC GAA CCA ACG CTC ATC CGT ATA TGG GGT
GAT GCT TTG TTT GAT ATC GTT GAC AAA GAT CAA AAT
GGA GCC ATT ACA CTG GAT GAA TGG AAA GCA TAC ACC
AAA GCT GCT GGT ATC ATC CAA TCA TCA GAA GAT TGC
GAG GAA ACA TTC AGA GTG TGC GAT ATT GAT GAA AGT
GGA CAA CTC GAT GTT GAT GAG ATG ACA AGA CAA CAT
TTA GGA TTT TGG TAC ACC ATG GAT CCT GCT TGC GAA
AAG CTC TAC GGT GGA GCT GTC CCC TAA.

This probe was obtained by inserting, in a eukaryotic expression vector (pcDNA3; 5.4 kb), the sequence encoding the p66shc protein condensed in frame with the sequence encoding aequorin.

The localization of the chimeric probe is determined by signal peptides contained inside the native sequence of the protein whose intracellular path is to be followed, in this specific case p66shc. The addition of the cDNA of aequorin to the C-terminal of the sequence encoding p66shc does not alter either the orientation or the physiological behaviour of the protein being examined.

At this point, transfection is effected of the chimeric protein probe obtained in different cellular lines, such as A431, DMS 79.

Engineering of the Cellular Line

In the example illustrated, it was not necessary for the cellular line used to express any other element to activate the cellular response. It is possible, however, depending on study requirements, to induce the cells to express a specific receptor for the molecule/drug to be tested in order to ensure an effective linkage degree, thus simplifying the functional analysis of the transduction systems.

Cellular Transfection

The cells cultivated on cellular culture flasks (75 cm²) were transfected with a vector containing the chimeric probe produced, using the most suitable transfection techniques for the cellular line in question. The stabilized transfected cellular line A431 was adopted as experimental model, using the “Effectene reagent” procedure (Qiagen, Germany), which guarantees a high percentage of positive cells for this cellular line.

24 hours after the transfection, the cells were maintained in a DMEM culture medium, without serum.

Collection of Cells Expressing the Chimeric Probe

36 hours after transfection, the cells contained in the flask were detached from the bottom by trypsinization. The cellular suspension was then transferred to a Falcon tube (15 to 50 ml) and subjected to centrifugation at 1200 rpm at 20° C. and finally the cellular precipitate was re-suspended in KRB (Krebs-Ringer modified buffer: 125 mM NaCl, 5 mM KCl, 1 mM Na₃PO₄, 1 mM MgSO₄, 5.5 mM glucose, 20 mM HEPES, pH 7.4, 37° C.).

Reconstitution of the Aequorin Photo-Protein to Active Form

The prosthetic group celentherazine was added to the suspension of cells expressing the p66shc-aequorin probe, at a final concentration of 5 μM.

Planting on Multi-Well Plates

50 μl of suspension containing transfected cells (corresponding to about 100,000 cells) were planted in each well of the plate. The cells were left to adhere for a time varying from 1 to 2 hours keeping them in the dark, due to the photo-instability of celentherazine.

Detection of the Response

At the end of the incubation period, the various molecules to be tested were added to each well and the plate with the treated cells was then put in direct contact with a photo-multiplier which measures the emission of photons on the part of aequorin.

Results

This simple test can be used for testing compounds capable of modulating the translocation of proteins of the shc family in live cells.

FIG. 6 shows a graphs which indicates the measuring of the translocation of p66shc, according to the method described above.

The examples shown were carried out on two parallel batches of cells:
a) A431 cells expressing the p66shc-aequorin (p66shc-AEQ) probe (dark lines).
These cells were in turn subdivided into:
A431 p66shc-aequorin cells treated with EGF (epidermic growth factor) (100 ng/ml) (thick dark line);
control A431 p66shc-aequorin cells, i.e. non treated with EGF (thin dark line);
b) A431 cells expressing the cytosol aequorin probe (cyt-AEQ) (not condensed to any protein) (light lines).
These cells were in turn subdivided into:
A431 cells with cytosol aequorin treated with EGF (100 ng/mL) (thick light line);
control A431 cells with cytosol aequorin, i.e. non treated with EGF (thin light line).

As can be observed from the trend of the graphs shown in FIG. 6, in the absence of external calcium, the emission of photons (expressed as cps: counts per second) has a very low intensity (lower than 100 cps), under all the conditions examined. Under such conditions, in fact, the concentration of calcium in the cytoplasm, where the cyt-AEQ probes (both in the absence and in the presence of EGF growth factor) and p66shc-AEQ probes (in the absence of EGF) are located, and below the plasmatic membrane, i.e. where the p66shc-AEQ probe translocates following treatment with EGF, is very low (0.1 μM).

The addition of external calcium induces a significant flow of this ion through the channels situated on the plasmatic membrane with a consequent increase in the concentration of calcium in the region below the plasmatic membrane. Under these conditions, an enormous increase in the emission of photons is registered (>100,000 cps) only in the cells expressing the p66shc-AEQ probe, treated with EGF, indicating the complete translocation of the protein.

In all the other groups of cells, there is no significant variation in the emission of photons (lower than 10,000 cps) on the part of aequorin.

From the graph, it is evident that all the cps values higher than those observed in the absence of the maximal activator (EGF) will indicate a complete translocation of the kinase. With this system, it is therefore possible to identify, on the one hand, the molecules capable of inducing a translocation of the shc proteins through alternative paths with respect to that activated by the EGF growth factor, and, on the other hand, those molecules capable of blocking the intracellular transduction path activated by the EGF factor, by verifying the completed or non-completed translocation of the protein.

It is also possible to determine the efficiency with which a molecule induces (or blocks) said translocation by expressing it on the basis of a range between the cps value obtained under control conditions (0%) (without EGF) and that obtained following maximal translocation (100%) (after treatment with EGF), which can be described as follows:
GOOD activator for values ranging from 70% to 100% with respect to the maximal activator;
MEDIUM activator for values ranging from 40% to 70% with respect to the maximal activator;
POOR activator for values ranging from 10% to 40% with respect to the maximal activator;
NON-activator for values below 10% with respect to the maximal activator.

This result clearly shows the validity of the system proposed for measuring the translocation of proteins from the cytosol compartment to the plasmatic membrane in response to external stimulations.

### EXAMPLE 4

### Analysis of the Variation in Concentration of a Second Cellular Messenger of Interest, cAMP

Materials and Methods

Construction of the mt-AEQ Probe

The description of the mt-AEQ probe is described in detail in the article (Rizzuto et al., 1992).

Engineering of the Cellular Expression Line of the mt-Aequorin Probe

For this application, two cellular lines were engineered to enable them to express two different chimeric receptors on which a different drugs capable of regulating the functions of cAMP, could be tested.

The first chimeric receptor used was constructed by condensing the extra-cellular portion of a beta adrenergic receptor (coupled with the production of cAMP) with the intra-cellular portion of an alpha adrenergic receptor (coupled with variations in the concentration of calcium) (Cotecchia et al., 1992) hereafter called beta/alpha adrenergic receptor.

Said receptor was expressed in HeLa cells thus obtaining an engineered cellular line capable of responding to stimulation with a certain drug of interest.

The second chimeric receptor used was constructed by condensing the extra-cellular portion of the ORL1 receptor for nociceptine (coupled with the production of cAMP) with the intracellular portion of an alpha adrenergic receptor (coupled with variations in the calcium concentration) (Cotecchia et al., 1992) hereafter called ORL1/alpha adrenergic receptor.

Said receptor was expressed in CHO cells, thus obtaining an engineered cellular line capable of responding to stimulation with a certain drug of interest.

Cellular Transfection

The cellular lines thus engineered were then both transfected with the vector containing the mt-AEQ probe by means of the calcium phosphate technique.

Collection of the Cells Expressing the Chimeric Probe

36 hours after the transfection, the cells, cultivated on flasks for cellular cultures (75 cm²), were detached from the bottom by trypsinization. The cellular suspension was then transferred to a Falcon tube (of 15 or 50 ml) and subjected to centrifugation at 1200 rpm in a cell centrifuge at 20° C. and finally the cellular precipitate was re-suspended in KRB/Ca²⁺ (Krebs-Ringer modified buffer: 125 mM NaCl, 5 mM KCl, 1 mM CaCl₂, 1 mM Na₃PO₄, 1 mM MgSO₄, 5.5 mM glucose, 20 mM HEPES, pH 7.4, 37° C.).

Reconstitution of the Aequorin Photo-Protein to Active Form

The prosthetic group celentherazine was added at a final concentration of 5 μM to the suspension of engineered cells expressing the mt-AEQ probe.

Planting on Multi-Well Plates

50 μl of suspension containing transfected cells (corresponding to about 100,000 cells) were planted in each well of the plate. The cells were left to adhere for a time varying from 1 to 2 hours keeping them in the dark, due to the photo-instability of celentherazine.

Detection of the Response

At the end of the incubation time, the various molecules to be tested were added to each well and the plate with the treated cells was then put in direct contact with a photo-multiplier which measures the emission of photons on the part of aequorin.

Results

This simple test can therefore be used for testing compounds capable of inducing, by the activation of specific receptors, variations in the concentration of cAMP.

The experiment was carried out on HeLa and CHO cells expressing the mt-AEQ probe and the chimeric receptor (beta/alpha adrenergic) and the chimeric receptor (ORL1/alpha adrenergic), respectively.

The HeLa cells (FIG. 7) were treated with:

Histamine (100 μM, SIGMA) to induce the normal increase in calcium due to the stimulation of the endogenous receptor for histamine Hi (dark line).

Isoproterenol (100 μM, SIGMA) to induce an increase in calcium due to the stimulation of the chimeric receptor (light line).

HeLa cells expressing a beta adrenergic receptor and stimulated with isoproterenol for inducing the stimulation of said receptor, were used as control (dotted line).

The CHO cells (FIG. 8) were treated with:

ATP (100 μM, SIGMA) to induce the normal increase in calcium due to the stimulation of the endogenous receptor for ATP P2Y (dark line).

Nociceptine (1 μM) to induce an increase in calcium due to the stimulation of the chimeric receptor (light line).

CHO cells expressing a wild-type ORL1 receptor and stimulated with nociceptine for inducing the stimulation of said receptor, were used as control (dotted line).

FIGS. 7 and 8 show the graphs which indicate two examples of the measurement of the production and variation in concentration of cAMP with the method described above. As can be observed from an analysis of the graphs, in the absence of stimulation, the emission of photons (cps: counts per second) is very low, almost null (100 cps). Under these basic conditions, the concentration of calcium in the mitochondrial matrix where the mt-AEQ probes are located, is extremely low (0.2 μM).

Stimulation with histamine, in the case of the HeLa cells, and with ATP, in the case of the CHO cells, induces a considerable and transient increase in the concentration of calcium in the mitochondrial matrix as widely described in literature (Rizzuto et al., 1992; Pinton et al., 1998).

This increase is reflected in an enormous emission of photons on the part of aequorin (from less than 20 photons emitted per second to over 80,000 photons).

In order to verify the validity of the system proposed, we stimulated the cells with a specific agonist of the extra-cellular portion of the chimeric receptor (isoproterenol and nociceptine). The light line shows how, also in this case, there is a significant increase in the emission of photons on the part of aequorin (from less than 20 photons emitted per second to over 80,000 photons) indicating that stimulation with isoproterenol and nociceptine (normally coupled with the production and increase in the concentration of cAMP) is converted into a signal linked to the variation in the concentration of calcium. On the contrary, the stimulation with isoproterenol of cells expressing a beta adrenergic receptor and the stimulation with nociceptine of cells expressing an ORL1 wild-type receptor, coupled therefore with the production of cAMP, does not induce any increase in cps, indicating the non-increase in concentration of calcium. It is evident that any substance which induces an increase in cps in cells expressing a chimeric receptor having the intracellular portion coupled with variations in the concentration of calcium, will be capable of modifying the concentration of cAMP.

This result clearly demonstrates the validity of the system proposed for measuring the activation of specific receptors coupled with the production of cAMP.

### EXAMPLE 5

### Analysis of the Activation/Inhibition of the Catalytic Activity of a Cellular Effector of Interest, PKC, in Controlling the Functional State of Channels for Calcium

In order to validate this method, analysis experiments were effected, of the catalytic activity of PKC on specific calcium channels situated at the level of the plasmatic membrane called L-type Ca²⁺ channels.

In this specific case, the cells were transfected with a Ca²⁺ channel of the L type, inhibited by a PKC-dependent phosphorylation, and with a probe represented by an aequorin localized below the plasmatic membrane (SNAP-AEQ) (Marsault et al., 1997), in direct contact with the channel being examined.

Materials and Methods

Construction of the SNAP-AEQ Probe

The description of the SNAP-AEQ probe is described in detail in the article (Marsault et al., 1997).

Engineering of the Cellular Expression Line of the SNAP-AEQ Probe

For this application, it is necessary to engineer a cellular line with the calcium channel (in this case of the L type).

The HeLa cells were engineered so as to express the calcium channel whose activity is regulated by the cellular effector of interest (PKC).

Cellular Transfection

The engineered cellular line was then transfected with the vector containing the SNAP-AEQ probe by means of the calcium phosphate method.

Collection of the Cells Expressing the Chimeric Probe

36 hours after the transfection, the cells, cultivated on flasks for cellular cultures (75 cm²), were detached from the bottom by trypsinization. The cellular suspension was then transferred to a Falcon tube (of 15 or 50 ml) and subjected to centrifugation at 1200 rpm, in a cell centrifuge at 20° C. and finally the cellular precipitate was re-suspended in KRB (Krebs-Ringer modified buffer: 125 mM NaCl, 5 mM KCl, 1 mM Na₃PO₄, 1 mM MgSO₄, 5.5 mM glucose, 20 mM HEPES, pH 7.4, 37° C.).

Reconstitution of the Aequorin Photo-Protein to Active Form

The prosthetic group celentherazine was added at a final concentration of 5 μM to the suspensions of engineered cells expressing the SNAP-AEQ probe.

Planting on Multi-Well Plates

50 μl of suspension containing the transfected and reconstituted cells (corresponding to about 100,000 cells) were planted in each well of the plate. The cells were left to adhere for a time varying from 1 to 2 hours keeping them in the dark, due to the photo-instability of celentherazine.

Detection of the Response

At the end of the incubation time, the various molecules to be tested were added to each well and the plate with the treated cells was then put in direct contact with a photo-multiplier which measures the emission of photons on the part of aequorin.

With this test it is possible to identify the molecules capable of modifying the catalytic activity of cellular effectors of interest and consequently the functional state of the calcium channels.

### BIBLIOGRAPHY

Brini, M., M. Murgia, L. Pasti, D. Picard, T. Pozzan, and R. Rizzuto. 1993. EMBO J. 12:4813-4819.
Brini, M., R. Marsault, C. Bastianutto, T. Pozzan, and R. Rizzuto. 1994a. Cell Calcium 16:259-268.
Brini, M., L. Pasti, C. Bastianutto, M. Murgia, T. Pozzan, and R. Rizzuto. 1994b. J. Biolumin. Chemilumin. 9:177-184.
Brini, M., R. Marsault, C. Bastianutto, J. Alvarez, T. Pozzan, and R. Rizzuto. 1995. J. Biol. Chem. 270:9896-9903.
Brini, M., F. De Giorgi, M. Murgia, R. Marsault, M. L. Massimino, M. Cantini, R. Rizzuto and T. Pozzan. 1997. Mol. Biol. Cell 8:129-143.
Brini, M., P. Pinton, T. Pozzan, and R. Rizzuto. 1999. Microsc. Res. Tech. 46:380-389.
Chiesa, A., E. Rapizzi, V. Tosello, P. Pinton, M. de Virgilio, K. E. Fogarty, and R. Rizzuto. 2001. Biochem. J. 355:1-12.
Cotecchia, S., F. Ostrowski, M. A. Kjelsberg, M. G. Caron, and R. J. Lefkowitz. 1992. J. Biol. Chem. 267:1633-1639.
De Giorgi, F., M. Brini, C. Bastianutto, R. Marsault, M. Montero, P. Pizzo, and R. Rizzuto. 1996. Gene 173:113-117.
Grynkiewicz. G., M. Poenie, and R. Y. Tsien. 1985. J. Biol. Chem. 260:3440-3450.
Marsault, R. M. Murgia, T. Pozzan, and R. Rizzuto. 1997. EMBO J. 16:1575-1581.
Montero, M., M. Brini, R. Marsault, J. Alvarez, R. Sitia, T. Pozzan, and R. Rizzuto. 1995. EMBO J. 14:5467-5475.
Pinton, P., M. Brini, C. Bastianutto, R. A. Tuft, T. Pozzan, and R. Rizzuto. 1998. Biofactors 8:243-253.
Pinton, P., T. Pozzan, and R. Rizzuto. 1998. EMBO J. 17:5298-5308.
Porcelli, A. M., P. Pinton, E. K. Ainscow, A. Chiesa, M. Rugolo, G. A. Rutter, and R. Rizzuto. 2001. Methods Cell Biol. 65:353-380.
Ridgway, E. B. and C. C. Ashley. 1967. Biochem. Biophys. Res. Commun. 29:229-234.
Rizzuto, R., A. W. Simpson, M. Brini, and T. Pozzan. 1992. Nature 358:325-327.
Rizzuto, R., M. Brini, and T. Pozzan. 1993. Cytotechnology 11 Suppl 1:S44-S46.
Rizzuto, R., M. Brini, and T. Pozzan. 1994. Methods Cell Biol. 40:339-358.
Rizzuto, R., M. Brini, C. Bastianutto, R. Marsault, and T. Pozzan. 1995. Methods Enzymol. 260:417-428.
Rizzuto, R., P. Pinton, W. Carrington, F. S. Fay, K. E. Fogarty, L. M. Lifshitz, R. A. Tuft, and T. Pozzan. 1998. Science 280:1763-1766.
Robert, V., P. Pinton, V. Tosello, R. Rizzuto, and T. Pozzan. 2000. Methods Enzymol. 327:440-456.
Rutter, G. A., P. Burnett, R. Rizzuto, M. Brini, M. Murgia, T. Pozzan, J. M. Tavare, and R. M. Denton. 1996. Proc. Natl. Acad. Sci. U.S.A. 93:5489-5494.
Shimomura, O., F. H. JOHNSON, and Y. SAIGA. 1962. J. Cell Comp Physiol 59:223-239.
Tsien, R. Y., T. Pozzan, and T. J. Rink. 1982. Nature 295:68-71.
Zhang, J., R. E. Campbell, A. Y. Ting, and R. Y. Tsien. 2002. Nat. Rev. Mol. Cell. Biol. 3:906-918

1-50. (canceled)

51. Screening method of molecules capable of generating the alteration of a target intracellular parameter, said alteration being the variation in the concentration of a second messenger that is a cyclic nucleotide, said alteration being converted into a proportional variation in the intracellular concentration of the Ca2+ ion, detected by means of a Ca2+-sensitive recombinant aequorin probe, comprising the following phases:
a) construction of an expression vector containing the fusion protein sequence encoding said probe, said sequence being characterized in that it comprises the Ca2+-sensitive recombinant aequorin encoding sequence, condensed together with at least one signal sequence;
b) transfection of at least one of a mammalian cell line with said vector containing the Ca2+-sensitive recombinant aequorin probe, said cell line being previously engineered so as to express an heterologous chimeric receptor being characterized in that it has the intracellular portion of a receptor coupled with variations in the concentration of calcium and the extra-cellular portion of a receptor coupled with the production of cyclic nucleotides;
c) activation of said Ca2+-sensitive aequorin probe by the addition of a prosthetic group to the cellular line expressing said recombinant protein probe;
d) administration of the molecule to be tested to the cellular line expressing said recombinant protein probe;
e) detection of the emission of photons on the part of the Ca2+-sensitive aequorin probe expressed in the cellular line and evaluate the amount of activation or inhibition exerted by the tested molecule, on the basis of a ratio between the cps value obtained and the maximum value of cps registered under conditions of maximum stimulation of the cellular line.

52. Screening method according to claim 51, wherein said prosthetic group is celentherazine.

53. Screening method according to claim 51, wherein said cyclic nucleotide is c-AMP.

54. Screening method according to anyone of the claim 51, wherein said signal sequence directs the Ca²⁺-sensitive recombinant aequorin probe to a cellular compartment, said probe being the fusion protein mt-aequorin (mt-AEQ).

55. Screening method of molecules capable of generating the alteration of a target intracellular parameter, said alteration being the translocation from cytoplasm to the membrane of a cellular effector, said translocation being correlated to the different intracellular concentration of the Ca2+ ion between cytoplasm and submembrane area, detected by means of a Ca2+-sensitive recombinant aequorin probe, comprising the following phases:
a) construction of an expression vector containing the fusion protein sequence encoding said probe, said sequence being characterized in that it comprises sequences encoding at least one Ca2+-sensitive recombinant aequorin encoding sequence, condensed together with at least one cellular effector and/or a signal sequence;
b) transfection of at least one mammalian cell line with said vector containing the Ca2+-sensitive recombinant protein probe;
c) activation of said Ca2+-sensitive photo-protein by the addition of a prosthetic group to the cellular line expressing said recombinant protein probe;
d) administration of the molecule to be tested to the cellular line expressing said recombinant protein probe;
e) detection of the emission of photons on the part of the Ca2+-sensitive photo-protein expressed in the cellular line and evaluate the amount of activation or inhibition exerted by the tested molecule, on the basis of a ratio between the cps value obtained and the maximum value of cps registered under conditions of maximum stimulation of the cellular line.

56. Screening method according to claim 55, wherein said prosthetic group is celentherazine.

57. Screening method according to claim 55, wherein said cellular effector is a regulating protein.

58. Screening method according to claim 57, wherein said regulating protein is selected from the group which comprises protein-kinase, phosphatase, adenylate cyclase, proteins that link plasmatic membrane receptors, proteins that interact with plasmatic membrane channels, proteins that interact with plasmatic membrane lipids.

59. Screening method according to claim 55, wherein said signal sequence directs the Ca2+-sensitive recombinant aequorin probe to a cellular compartment.

60. Screening method according to claim 55, wherein said probe is a fusion protein selected from the group that consists of PKC-aequorin (PKC-AEQ) and shc-aequorin (shc-AEQ).

61. Screening method according to claim 60, wherein the PKC-aequorin is selected from the group comprising PKC beta-aequorin, PKC delta-aequorin, PKC epsilon-aequorin, PKC zeta-aequorin, PKC gamma-aequorin, PKC alpha-aequorin, PKC-lambda-aequorin, PKC theta-aequorin, PKC eta-aequorin.

62. Screening method according to claim 60, wherein the shc-aequorin is selected from the group consisting of p66shc-aequorin, p46shc-aequorin, p52shc-aequorin.

63. Screening method according to claim 55, wherein the expression vector of phase a) is a eukaryotic vector.

64. Screening method according to claim 55, wherein said at least one mammal cellular line of phase b) is previously engineered so as to express a heterologous native or chimeric protein.

65. Screening method according to claim 64, wherein said heterologous protein is selected from the group which consists of a receptor, an enzyme, a ionic channel and a cellular effector.

66. Screening method according to claim 64, wherein said chimeric protein is a chimeric receptor.

67. Screening method according to claim 65, wherein said ionic channel is selected from the group which comprises voltage-dependent Ca2+ channels and Ca²⁺ channel receptors.

68. Screening method according to claim 65, wherein said cellular effector is a regulating protein selected from the group which comprises protein-kinase, phosphatase, adenylate cyclase, proteins that links plasmatic membrane receptors, proteins that interacts with plasmatic membrane channels, proteins that interacts with plasmatic membrane lipids.

69. Screening method according to claim 65, wherein said cellular effector is a cell membrane receptor selected from the group which comprises receptors coupled with G proteins, receptors with an enzymatic activity, channel receptors.

70. A Ca2+-sensitive recombinant fusion protein probe, characterized in that it comprises the sequence encoding at least one Ca2+-sensitive recombinant aequorin encoding sequence, condensed together with at least one cellular effector and/or a signal sequence, said cellular effector being a regulating protein selected from a protein-kinase and a protein that links plasmatic membrane receptors.

71. Probe according to claim 70, wherein said protein-kinase is a protein kinase C (PKC).

72. Probe according to claim 71, wherein said PKC-aequorin is selected from the group which comprises PKC beta-aequorin (PCK beta: rif. M13975), PKC delta-aequorin (PCK delta: rif. M18330), PKC epsilon-aequorin (PCK epsilon: rif. AF028009), PKC zeta-aequorin (PCK zeta: rif. M18332), PKC gamma-aequorin, PKC alpha-aequorin (PCK alfa: rif. M13973), PKC-lambda-aequorin, PKC theta-aequorin (PCK theta: rif. L07032), PKC eta-aequorin.

73. Probe according to claim 70, wherein said protein that links plasmatic membrane receptors is an adaptor protein.

74. Probe according to claim 73, wherein said adaptor protein belongs to the shc family.

75. Probe according to claim 74, wherein the protein is selected from the group comprising p46shc, p52shc and p66shc.

76. Probe according to claim 70, wherein said cell membrane receptor is selected from the group which comprises receptors coupled with G proteins, receptors with an enzymatic activity, channel receptors.

77. Probe according to claim 70, wherein said signal sequence directs the Ca2+-sensitive photo-protein, preferably aequorin, towards a cellular compartment.

78. Use of the Ca2+-sensitive recombinant fusion protein probe as defined in claim 70, for the screening of molecules capable of generating the alteration of an intracellular parameter, said alteration being the translocation from cytoplasm to the membrane of a cellular effector, said translocation being correlated to the different intracellular concentration of the Ca2+ ion between cytoplasm and submembrane area.

79. Use according to claim 78, wherein said cellular effector is selected from the group which consists of ionic channel, regulating protein, cell membrane receptor.

80. Use according to claim 79, wherein said ionic channel is selected from the group which comprises voltage-dependent Ca2+ channels and 2+ channel receptors.

81. Use according to claim 79, wherein said regulating protein is selected from the group which comprises protein-kinase, phosphatase, adenylate cyclase, proteins that links plasmatic membrane receptors, proteins that interacts with plasmatic membrane channels, proteins that interacts with plasmatic membrane lipids.

82. Use according to claim 79, wherein said cell membrane receptor is selected from the group which comprises receptors coupled with G proteins, receptors with an enzymatic activity, channel receptors.

83. Screening method of molecules capable of generating the alteration of a target intracellular parameter, said alteration being the activation/inactivation of a cellular effector that is a protein kinase acting on Ca2+ ionic channels, said alteration being converted into a proportional variation in the intracellular concentration of the Ca2+ ion, detected by means of a Ca2+-sensitive recombinant aequorin probe, comprising the following phases:
a) construction of an expression vector containing the fusion protein sequence encoding said probe, said sequence being characterized in that it comprises the Ca2+-sensitive recombinant aequorin encoding sequence, condensed together with at least one signal sequence;
b) transfection of at least one of a mammalian cell line with said vector containing the Ca2+-sensitive recombinant aequorin probe;
c) activation of said Ca2+-sensitive aequorin probe by the addition of a prosthetic group to the cellular line expressing said recombinant protein probe;
d) administration of the molecule to be tested to the cellular line expressing said recombinant protein probe;
e) detection of the emission of photons on the part of the Ca2+-sensitive aequorin probe expressed in the cellular line and evaluate the amount of activation or inhibition exerted by the tested molecule, on the basis of a ratio between the cps value obtained and the maximum value of cps registered under conditions of maximum stimulation of the cellular line.

84. Screening method according to claim 83, wherein said prosthetic group is celentherazine.

85. Screening method according to claim 83, wherein said cellular effector is PKC.

86. Screening method according to claim 83, wherein said Ca2+ ionic channel is selected from the group which comprises voltage dependent Ca2+ channels and Ca2+ channel-receptors.

87. Screening method according to claim 86, wherein said Ca2+ channels are L type Ca2+ channels.

88. Screening method according to claim 83, wherein said signal sequence directs the Ca2+-sensitive recombinant aequorin probe to a cellular compartment.

89. Screening method according to claim 83, said probe being a fusion protein selected from the group which comprises SNAP aequorin (SNAP-AEQ), mt-aequorin (mt-AEQ) and cytosol aequorin (cyt-AEQ).

90. Screening method according to claim 83, wherein said at least one mammal cellular line of phase b) is previously engineered so as to express a heterologous native or chimeric protein.

91. Screening method according to claim 90, wherein said heterologous protein is selected from the group which consists of ionic channels.

92. Screening method according to claim 91, wherein said ionic channel is selected from the group which comprises voltage-dependent Ca2+ channels and Ca2+ channel receptors.

93. Screening method according to claim 92, wherein said Ca2+ channels are L type Ca2+ channels.
